(19) United States
(12) Patent Application Publication
(10) Pub. No.: US 2017/0367712 A1
(43) Pub. Date: Dec. 28, 2017

(54) STRETCH-RESISTANT COIL

(71) Applicant: Boston Scientific Scimed, Inc., Maple Grove, MN (US)

(72) Inventors: Jeffry D. Johnson, Crystal, MN (US); Nicholas L. Tassoni, Andover, MA (US); Ken X. Zhang, Maple Grove, MN (US)

(21) Appl. No.: 15/631,129

(22) Filed: Jun. 23, 2017

Related U.S. Application Data
(60) Provisional application No. 62/354,152, filed on Jun. 24, 2016.

(51) Int. Cl. A61B 17/12

(52) U.S. Cl. CPC A61B 17/12154; A61B 17/1214; A61B 17/12022; A61B 2017/1205

(57) ABSTRACT

The present disclosure relates to the field of delivery systems for precise navigation within and through body passages. Specifically, the present disclosure relates to delivery systems for accurate positioning and release of elements within tortuous, narrow and/or fragile passages. In particular, the present disclosure relates to a delivery system that includes a distal coil with sufficient flexibility to navigate through tortuous body passages, and which allows for controlled stretch when bent.

### CROSS-REFERENCE TO RELATED APPLICATIONS

This application claims the benefit of U.S. Provisional Application No. 62/354,152, entitled “STRETCH-RESISTANT COIL” and filed Jun. 24, 2016, which is hereby incorporated by reference in its entirety.

### FIELD

The present disclosure relates to the field of delivery systems for precise navigation within and through body passages. Specifically, the present disclosure relates to delivery systems for accurate positioning and release of elements within tortuous, narrow and/or fragile passages, e.g., occlusive elements within the vasculature. In particular, the present disclosure relates to a delivery system that includes a distal coil with sufficient flexibility to navigate through tortuous body passages, and which allows for controlled stretch when bent, e.g., to prevent premature release in the case of an occlusive element.

### BACKGROUND

A variety of delivery systems are available for minimally invasive medical procedures which require precise navigation of body passages to administer therapeutic treatments, deliver medical device (e.g., stents, grafts, occlusive elements etc.), retrieve biopsy samples, visualize and remove obstructions (e.g., blood clots, plaque, bowel obstructions etc.), and visualize unhealthy or potentially unhealthy tissues. Virtually all body passageways include some sort of internal architecture (e.g., narrow strictures; sharp turns or bifurcation points; thin, fragile or damaged tissue walls; proximity to vital nerves, organs or vessels etc.) which require delicate and precise maneuvering of the delivery system to avoid unintended internal damage. For example, proper positioning and delivery of occlusive elements within a target site, such as an aneurysm, requires a delivery system capable of navigating the narrow and tortuous passages of the vasculature system. This is often achieved with a delivery system that includes a flexible coil on its distal end. These coils require sufficient rigidity to provide the “pushability” necessary to maintain their linear configuration within the vasculature (i.e., resist buckling), while also providing the requisite “steerability” (i.e., flexibility) to guide the occlusive element through vascular junctions and/or curvatures.

Although conventional delivery systems generally include a flexible coil tip that provides the necessary “pushability” and “steerability,” such coils tend to increase in length (i.e., stretch) upon bending as adjacent coil windings separate along the leading edge. In many instances, the most dramatic bending of the distal coil, and therefore most significant stretching, occurs as the delivery system pivots for final positioning of the occlusive element within the target site. Stretching may also occur if the delivery system is retracted proximally within the vasculature while the coil tip and/or occlusive element is disposed within a narrow (i.e., high-friction) portion of the vasculature. Lengthening of the coil tip may force the occlusive element to prematurely release from the delivery system. Prematurely released occlusive elements s may result in a variety of negative medical outcomes, including partial occlusion of the target site and/or occlusion of vital non-target vascular passageways. Retrieval of improperly deployed occlusive elements, to the extent possible, results in longer procedure times and increases the likelihood of additional complications.

There is an ongoing need for an occlusive element delivery system that includes a distal coil with the requisite “pushability” and “steerability” to allow precise navigation and positioning, but which does not stretch when bent or retracted proximally.

### SUMMARY

The present disclosure, in its various aspects, meets an ongoing need in the medical field, such as the field of occlusive element 1 delivery, for a delivery system that includes a stretch-resistant coil to prevent premature release of the occlusive element.

In one aspect, the present disclosure provides a wound wire comprising a plurality of adjacent windings, wherein the wound wire includes a first region, a second region, and an intermediate region between the first and second regions, and wherein at least a portion of the windings of the intermediate region include a differential welding pattern between one or more adjacent windings along a length thereof. The differential welding pattern may impart stretch resistance to at least a portion of the intermediate region. The differential welding pattern may impart a first plane of restricted flexibility to a first pair of adjacent windings, and a second plane of restricted flexibility to a second pair of adjacent winding. The first plane of restricted flexibility may be perpendicular to the second plane of restricted flexibility. The first and second planes of restricted flexibility may be on adjacent windings. Alternatively, the first and second planes of restricted flexibility may not be on adjacent windings. Each winding of the intermediate region may include opposing first and second welds, wherein opposing welds on adjacent coil windings are offset approximately 90°. Alternatively, alternating winds of the intermediate region may include opposing first and second welds, wherein opposing welds on alternating adjacent windings are offset approximately 90°. The first region, second region and intermediate region may include the same number of windings. Alternatively, the first region may include more windings than either the intermediate region or the second region. Alternatively, the intermediate region may include fewer windings than the first region, and more windings than the second region. The intermediate region may include at least 3 windings, at least 5 windings, or at least 7 windings. The wire may be formed from a metal comprising platinum, tungsten, titanium, stainless steel, nickel rhodium, palladium, rhenium, gold, silver, tantalum, and alloys of these metals including platinum/tungsten alloys and nickel-titanium alloys.

In another aspect, the present disclosure provides a coil, comprising a wound wire including a plurality of adjacent windings, wherein the wound wire may include a first region, a second region, and an intermediate region between the first and second regions. At least a portion of the windings of the intermediate region may include a differential welding pattern between one or more adjacent windings along a length thereof. The differential welding pattern may impart a first plane of restricted flexibility to a first pair of adjacent windings and a second plane of restricted flexibility to a second pair of adjacent windings, wherein the first and second planes of restricted flexibility are on adjacent windings. The differential welding pattern may impart stretch resistance to at least a portion of the intermediate region. The first plane of restricted flexibility may be perpendicular to the second plane of restricted flexibility. The first region, second region and intermediate region may include the same number of windings. The first region may include more windings than either the intermediate region or the second region. The intermediate region may include fewer windings than the first region, and more windings than the second region. The wound wire may be formed from a metal comprising platinum, tungsten, titanium, stainless steel, nickel rhodium, palladium, rhenium, gold, silver, tantalum, and alloys of these metals including platinum/tungsten alloys and nickel-titanium alloys.

In yet another aspect, the present disclosure provides a coil, comprising a wound wire including a plurality of adjacent windings, wherein the wound wire may include a first region, a second region, and an intermediate region between the first and second regions. At least a portion of the windings of the intermediate region may include a differential welding pattern between one or more adjacent windings along a length thereof. The differential welding pattern may impart a first plane of restricted flexibility to a first pair of adjacent windings and a second plane of restricted flexibility to a second pair of adjacent windings, wherein the first and second planes of restricted flexibility are not on adjacent windings. The differential welding pattern may impart stretch resistance to at least a portion of the intermediate region. The first plane of restricted flexibility may be perpendicular to the second plane of restricted flexibility. The first region, second region and intermediate region may include the same number of windings. The first region may include more windings than either the intermediate region or the second region. The intermediate region may include fewer windings than the first region, and more windings than the second region. The wound wire may be formed from a metal comprising platinum, tungsten, titanium, stainless steel, nickel rhodium, palladium, rhenium, gold, silver, tantalum, and alloys of these metals including platinum/tungsten alloys and nickel-titanium alloys.

In yet another aspect, the present disclosure provides a delivery system, comprising an elongate flexible pusher member including a proximal end, a distal end, and a lumen extending therethrough. A coil may be coupled to the distal end of the pusher member. The coil may include a first region, a second region, and an intermediate region between the first and second regions, wherein the intermediate region exhibits greater resistance to stretching than the first and second regions. A retaining member may be coupled to a distal portion of the coil, and a releasable element releasably may be coupled to a distal portion of the retaining member. The first region of the coil may be fixedly disposed within the lumen of the distal end of the pusher member by one of a weld, solder, adhesive, glue or resin. A proximal portion of the retaining member may be fixedly disposed within a lumen of the second region of the coil by one of a weld, solder, adhesive, glue or resin. The retaining member may include a lumen extending therethrough, and the lumen of the retaining member may include a gripping element. The releasable element may include an attachment member configured to reversibly engage the gripping element of the retaining member. The delivery system may further include an elongate filament that extends along a length of the elongate flexible pusher member and coil. The elongate filament may include a distal end that engages a portion of the releasable element. Retracting the elongate filament in a proximal direction relative to the elongate flexible pusher member may disengage the filament from the releasable element, thereby releasing the releasable element. The stretch-resistant coil may be formed from a metal comprising platinum, tungsten, titanium, stainless steel, nickel rhodium, palladium, rhenium, gold, silver, tantalum, and alloys of these metals including platinum/tungsten alloys and nickel-titanium alloys. The elongate pusher member may be formed from a polymer such as acrylate-based polymers, polyurethane-based polymers, polynorbornene-based polymer and polylactide-based polymers. The elongate push catheter may include an outer diameter of about 2.0 millimeters. The stretch-resistant coil may include an outer diameter of about 2.0 millimeters.

### BRIEF DESCRIPTION OF THE DRAWINGS

Non-limiting examples of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of skill in the art to understand the disclosure. In the figures:

FIGS. 1A-1B illustrate side (FIG. 1A) and expanded (FIG. 1B) views of a stretch-resistant coil, according to an embodiment of the present disclosure.

FIGS. 2A-2B illustrate side (FIG. 2A) and expanded (FIG. 2B) views of a delivery system for an occlusive element that includes a distal stretch-resistant coil, according to an embodiment of the present disclosure.

It is noted that the drawings are intended to depict only typical or exemplary embodiments of the disclosure. Accordingly, the drawings should not be considered as limiting the scope of the disclosure. The disclosure will now be described in greater detail with reference to the accompanying drawings.

### DETAILED DESCRIPTION

Before the present disclosure is described in further detail, it is to be understood that the disclosure is not limited to the particular embodiments described, as such may vary. It is also to be understood that the terminology used herein is for the purpose of describing particular embodiments only, and is not intended to be limiting beyond the scope of the appended claims. Unless defined otherwise, all technical terms used herein have the same meaning as commonly understood by one of ordinary skill in the art to which the disclosure belongs. Finally, although embodiments of the present disclosure are described with specific reference to delivery systems that include a flexible and stretch-resistant distal coil for precise delivery of occlusive elements, it should be appreciated that such stretch-resistant coils may be used in a variety of navigation and delivery system to access, e.g., the upper and lower gastrointestinal tracts, respiratory system, nervous system, uterine artery and fallopian tubes etc.

As used herein, the term “distal” refers to the end farthest away from a medical professional when introducing a device into a patient, while the term “proximal” refers to the end closest to the medical professional when introducing a device into a patient.

As used herein, the term “weld” refers to the joining together of two or more pieces (or different portions of a single piece). In one embodiment, a weld may be formed by applying extreme heat to a metal or thermoplastic material such that the materials fuse together. In another embodiment, a weld may be formed using lower temperature techniques such as soldering or brazing, which do not melt the base material. In yet another embodiment, a weld may be formed using suitable adhesives (i.e., glue etc.) or fasteners (i.e., clips, clamps, knots etc.).

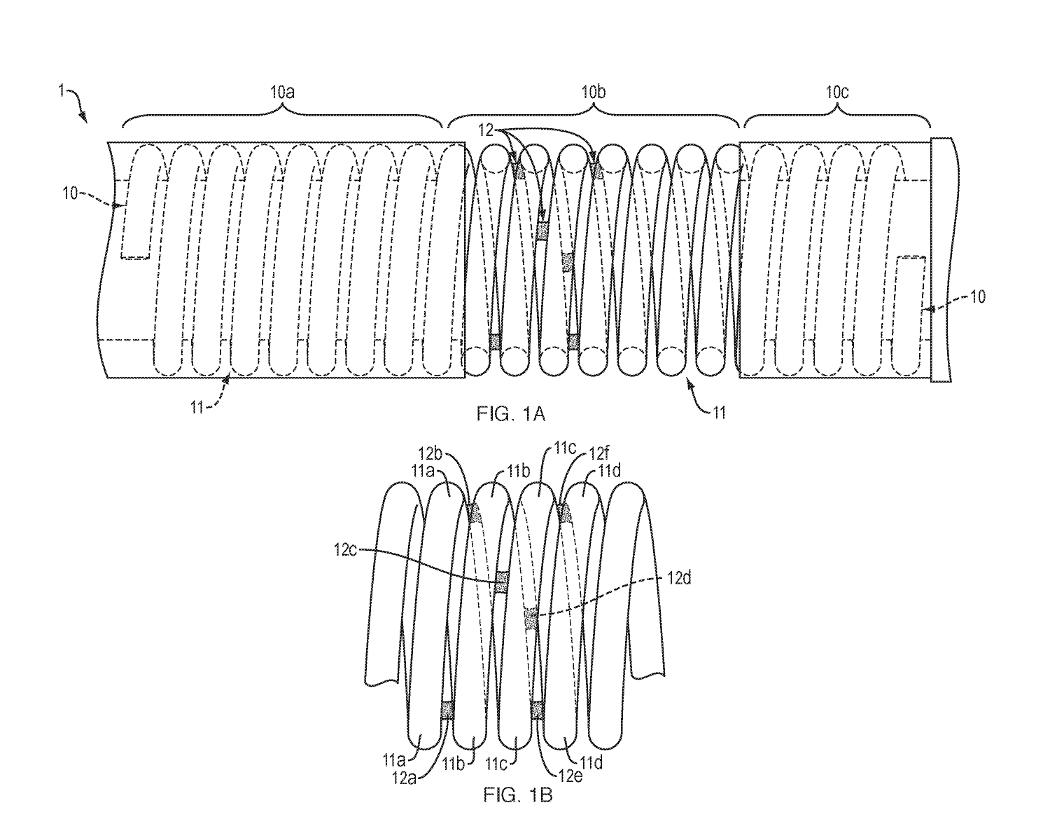

In one embodiment, the present disclosure provides a stretch-resistant coil configured to provide the requisite flexibility and “pushability” to navigate to a target site through the vasculature, but which does not stretch when bent. As illustrated in FIG. 1A, the stretch-resistant coil 1 of the present disclosure may include a helically wound wire 10 comprising a plurality of adjacent windings 11 generally arranged in a first region 10a, a second region 10c and an intermediate region 10b between the first and second regions. Although the stretch-resistant coil depicted in FIG. 1A includes approximately twenty windings, it will be appreciated that stretch-resistant coils of the present disclosure may include fewer than twenty windings (i.e., 19 or fewer windings), or more than twenty winding (i.e., 21 or more windings). It should also be appreciated that the number of windings 11 within the first region 10a, second region 10c and intermediate region 10b may vary. For example, the stretch-resistant coil 1 depicted in FIG. 1A includes approximately eight windings in the first region 10a, approximately eight windings in the intermediate region 10b, and approximately four windings in the second region 10c. In some embodiments, the first region 10a may include a greater number of windings than either of the second region 10c and intermediate region 10b. In other embodiments, the intermediate region 10b may include a greater number of windings than either of the first region 10a and second region 10c. In yet another embodiment, the second region 10c may include a greater number of windings than either of the first 10a region and intermediate region 10b.

Still referring to FIG. 1A, the adjacent windings 11 of the intermediate region 10b may be attached by a plurality of welds 12 arranged in a differential pattern along the coil's length. For example, referring to FIG. 1B, adjacent windings 11a and 11b may be fused together by welds 12a, 12b on opposite sides of the windings (i.e., 180° of separation), adjacent windings 11b and 11c may be fused together by opposing welds 12c, 12d (i.e., 180° of separation) offset by approximately 90° from welds 12a and 12b, and adjacent windings 11c and 11d may be fused together by opposing welds 12e, 12f (i.e., 180° of separation) offset by approximately 90° from welds 12c, 12d and approximately aligned with welds 12a, 12b. As will be understood by those of skill in the art, this differential welding pattern significantly limits linear separation (i.e., stretching) of the intermediate region 10b when the stretch-resistant coil 1 is bent or pulled, while only minimally reducing flexibility by restricting bending of adjacent windings along a single plane. For example, the welding pattern of FIGS. 1A-1B provides alternating perpendicular planes of restricted flexibility in which adjacent windings 11a and 11b are restricted in a first plane of flexibility X, adjacent windings 11b and 11c are restricted in a second plane of flexibility Y perpendicular to the first plane of flexibility X, and adjacent windings 11c and 11d are restricted in the first plane of flexibility X.

It should be appreciated that the welding pattern outlined above may be repeated along intermediate region 10b as necessary to achieve the desired flexibility and stretch resistance. In one embodiment, adjacent windings are fused along the entire length of intermediate region 10b. In another embodiment, adjacent windings are fused along a portion of the intermediate region 10b, while other adjacent windings of the intermediate region 10b remain unfused. In yet another embodiment, one or more unfused windings may be interspersed between fused adjacent windings.

It should also be appreciated that adjacent windings of the coil may be fused together using a differential welding pattern which provides greater than two alternating perpendicular planes of flexibility. For example, adjacent windings may be fused by opposing welds (i.e., 180° of separation) which are offset by approximately 120° from the next pair of opposing welds (i.e., 180° of separation). This welding pattern may be repeated along three sets of adjacent windings to provide an intermediate region which is more restricted in its ability to linearly separate than the coil of FIGS. 1A-1B, and which includes three separate planes (X, Y, Z) of restricted flexibility. In one embodiment, adjacent windings are fused along the entire length of intermediate region using this welding pattern. In another embodiment, adjacent windings are fused along a portion of the intermediate region using this welding pattern, while other adjacent windings of the intermediate region 10b remain unfused. In yet another embodiment, one or more unfused windings may be interspersed between fused adjacent windings using this welding pattern. The degree of offset between opposing welds (i.e., 180° of separation) may be varied (e.g., approximately 170° of separation, approximately 160° of separation, approximately 150° of separation, approximately 140° of separation, approximately 130° of separation, approximately 110° of separation, approximately 100° of separation, approximately 90° of separation or less) depending on the number of planes of flexibility desired.

It should be further appreciated that adjacent windings of the coil may be fused together using a differential welding pattern which provides a single plane of restricted flexibility. For example, adjacent windings may be fused by opposing welds (i.e., 180° of separation) which are offset by approximately 180° from the next pair of opposing welds (i.e., 180° of separation). This welding pattern may be repeated along three sets of adjacent windings to provide an intermediate region which is less restricted in its ability to linearly separate than the coil of FIGS. 1A-1B, and which includes a single plane (X) of restricted flexibility.

It should also be appreciated that the present disclosure is not limited to adjacent windings which are fused by opposing welds, but may include welds positioned at fixed intervals along one or more windings of the intermediate region. For example, nine welds may be positioned every 40° along one complete winding (e.g., 360°) of the intermediate region. This pattern of welds may be repeated on all or a portion of the adjacent windings of the intermediate region to satisfy the performance characteristics of a given medical procedure. Additionally, the pattern of welds along the intermediate region may be interspersed within one or more windings that do not include any welds. The fixed interval of welds per complete winding of the intermediate region is not limited to 40°, but may include any number of evenly spaced welds (e.g., welds approximately every 30°, approximately every 50°, approximately every 60°, approximately every 70°, approximately every 80°, approximately every 90°, approximately every 100°, approximately every 110° and combinations therebetween).

Alternatively, adjacent windings of the coil may be fused together using a differential welding pattern in which adjacent welds are separated by 360°, e.g., all welds are positioned on the same side of the coil. This welding pattern may be repeated along three sets of adjacent windings to provide an intermediate region which is less restricted in its ability to linearly separate than the coil of FIGS. 1A-1B, and which is restricted in flexibility along one side of plane X (i.e., the side of the coil opposite the welds) but is not substantially restricted in flexibility along the opposite side of plane X (i.e., full flexibility on the opposite plane (i.e., the side of the coil that includes the welds.)

The wire 10 of the stretch-resistant coil 1 may include a variety of kink resistant materials, including, for example, platinum, tungsten, titanium, stainless steel, nickel rhodium, palladium, rhenium, gold, silver, tantalum, and alloys of these metals including platinum/tungsten alloys and nickel-titanium alloys. The stretch-resistant coil 1 of the present disclosure may include a variety of lengths corresponding to the anatomical location of the target site. By way of non-limiting example, the length of the stretch-resistant coil 1 may be from about 0.5 centimeters to about 200 centimeters; and more preferably from about 20 centimeters to about 50 centimeters. The stretch-resistant coil 1 may also include an outer diameter from about 0.25 millimeters to about 5.0 millimeters; and more preferably from about 1.0 millimeters to about 1.5 millimeters.

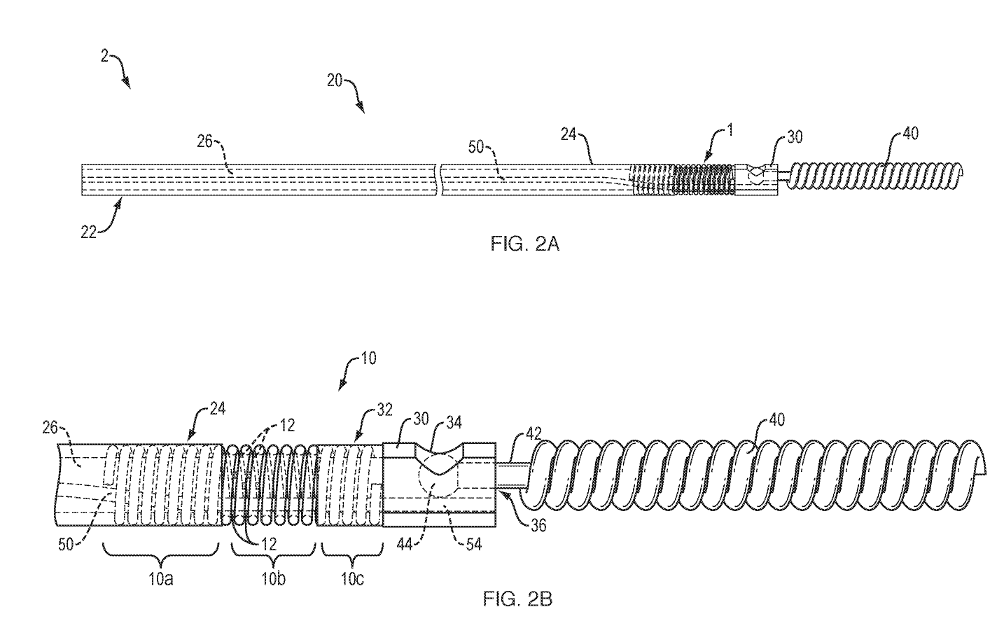

Referring to FIG. 2A, the present disclosure may include a delivery system 2 comprising an elongate flexible pusher member 20 with a proximal end 22, distal end 24 and lumen 26 extending therebetween. The distal end 24 of the pusher member 20 may be fixedly coupled to a proximal portion of the stretch-resistant coil 1 (as described in FIG. 1A), and a retaining member 30 may be fixedly coupled to a distal portion of the stretch resistant coil. A releasable element 40 (e.g., embolic coil) may be releasably coupled to the retaining member 30. The delivery system 2 may further include an elongate filament 50 that extends along the length of the lumen 26 of the pusher member 20, through an interior portion of the helically wound wire 10 and into a lumen 36 of the retaining member 30.

The elongate flexible pusher member 20 may be formed from a variety of flexible/bendable polymers comprising, for example, nylon (e.g., such as nylon 12, nylon 11, nylon 6/12, nylon 6, nylon 66), polyesters (e.g., polyethylene terephthalate (PET), polybutylene terephthalate (PBT), polyethylene naphthalate (PEN), polytrimethylene terephthalate (PTT); polyethers; polyurethanes; polyvinyls; polyacrylics; fluoropolymers; copolymers and block copolymers thereof, such as block copolymers of polyether and polyamide (e.g., PEBAX®); and mixtures thereof.

As best illustrated in FIG. 2B, the first region 10a of the helically wound wire 10 may be fixedly disposed within the lumen 26 at the distal end 24 of the elongate flexible pusher member 20. Similarly, a proximal end 32 of the retaining member 30 may be fixedly disposed within an interior portion of the second region 10c of the helically wound wire 10. As will be understood of those in the art, the first region 10a may be attached to an inner surface of the elongate flexible pusher member 20, and the second region 10c may be attached to the proximal end 32 of the retaining member 30 by one of a weld, solder, adhesive, glue or resin.

The lumen 36 of the retaining member 30 may include a gripping element 34 (e.g., socket, tab, flange etc.) configured to reversibly engage an attachment member 44 (e.g., ball-tip etc.) on the proximal 42 of the releasable element 40. A distal end 54 of the elongate filament 50 may frictionally engage an outer surface of the attachment member 44 to maintain an interference fit between the attachment member 44 and gripping element 34. The elongate filament 50 may be retracted in a proximal direction relative to the elongate flexible pusher member 20 such that the distal end 54 is removed from contact with (i.e., disengages) the attachment member 44, thereby releasing the releasable element 40 (not depicted).

It should be appreciated that the retaining member, releasable element and elongate filament described herein represent non-limiting examples of detachment mechanisms and/or releasable elements amenable for use with the stretch-resistant coil of the present disclosure. Finally, although the embodiments of the present disclosure have been described in use with an occlusive element attached to a stretch-resistant coil on the distal end of an elongate flexible pusher member, it should be appreciated that the delivery system may further include a retractable sheath disposed over at least a portion of the stretch-resistant coil and occlusive element. Such a sheath may provide a variety of useful purposes, such as protecting the occlusive element during delivery (e.g. preventing bending and/or agglomeration with body fluids), and preventing asymmetric surfaces of the stretch-resistant coil, retaining member and/or releasable element from abrading or otherwise damaging the surfaces of the vascular passageway.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

What is claimed is:

1. A coil, comprising:
a wound wire comprising a plurality of adjacent windings, wherein the wound wire includes a first region, a second region, and an intermediate region between the first and second regions,
wherein at least a portion of the windings of the intermediate region include a differential welding pattern between one or more adjacent windings along a length thereof,
wherein the differential welding pattern imparts a first plane of restricted flexibility to a first pair of adjacent windings and a second plane of restricted flexibility to a second pair of adjacent windings, and
wherein the first and second planes of restricted flexibility are on adjacent windings.

2. The coil of claim 1, wherein the differential welding pattern imparts stretch resistance to at least a portion of the intermediate region.

3. The coil of claim 1, wherein the first plane of restricted flexibility is perpendicular to the second plane of restricted flexibility.

4. The coil of claim 1, wherein the first region, second region and intermediate region include the same number of windings.

5. The coil of claim 1, wherein the first region includes more windings than either the intermediate region or the second region.

6. The coil of claim 1, wherein the intermediate region includes fewer windings than the first region, and more windings than the second region.

7. The coil of claim 1, wherein the wound wire is formed from a metal comprising platinum, tungsten, titanium, stainless steel, nickel rhodium, palladium, rhenium, gold, silver, tantalum, and alloys of these metals including platinum/tungsten alloys and nickel-titanium alloys.

8. A coil, comprising:
a wound wire comprising a plurality of adjacent windings, wherein the wound wire includes a first region, a second region, and an intermediate region between the first and second regions,
wherein at least a portion of the windings of the intermediate region include a differential welding pattern between one or more adjacent windings along a length thereof,
wherein the differential welding pattern imparts a first plane of restricted flexibility to a first pair of adjacent windings and a second plane of restricted flexibility to a second pair of adjacent windings, and
wherein the first and second planes of restricted flexibility are not on adjacent windings.

9. The coil of claim 8, wherein the differential welding pattern imparts stretch resistance to at least a portion of the intermediate region.

10. The coil of claim 8, wherein the first plane of restricted flexibility is perpendicular to the second plane of restricted flexibility.

11. The coil of claim 8, wherein the first region, second region and intermediate region include the same number of windings.

12. The coil of claim 8, wherein the first region includes more windings than either the intermediate region or the second region.

13. The coil of claim 8, wherein the intermediate region includes fewer windings than the first region, and more windings than the second region.

14. A delivery system, comprising:
an elongate flexible pusher member, comprising:
a proximal end,
a distal end, and
a lumen extending therethrough;
a coil coupled to the distal end of the pusher member, the coil comprising:
a first region,
a second region, and
an intermediate region between the first and second regions, wherein the intermediate region exhibits greater resistance to stretching than the first and second regions;
a retaining member coupled to a distal portion of the coil; and
a releasable element releasably coupled to a distal portion of the retaining member.

15. The delivery system of claim 14, wherein the first region of the coil is fixedly disposed within the lumen of the distal end of the pusher member by one of a weld, solder, adhesive, glue or resin.

16. The delivery system of claim 14, wherein a proximal portion of the retaining member is fixedly disposed within a lumen of the second region of the coil by one of a weld, solder, adhesive, glue or resin.

17. The delivery system of claim 14, wherein the retaining member includes a lumen extending therethrough, and wherein the lumen of the retaining member includes a gripping element.

18. The delivery system of claim 17, wherein the releasable element includes an attachment member configured to reversibly engage the gripping element of the retaining member.

19. The delivery system of claim 14, further comprising an elongate filament that extends along a length of the elongate flexible pusher member and coil, the elongate filament comprising a distal end that engages a portion of the releasable element.

20. The delivery system of claim 19, wherein retracting the elongate filament in a proximal direction relative to the elongate flexible pusher member disengages the filament from the releasable element, thereby releasing the releasable element.
